(19) United States
(12) Patent Application Publication
(10) Pub. No.: US 2016/0262688 A1
(43) Pub. Date: Sep. 15, 2016

(54) SYSTEM AND APPARATUS FOR POSTURE AND BODY POSITION CORRECTION AND IMPROVEMENT THROUGH A COMPUTER-ASSISTED BIOFEEDBACK SYSTEM

(71) Applicant: Andrew Nichols, Coconut Grove, FL (US)

(72) Inventor: Andrew Nichols, Coconut Grove, FL (US)

(21) Appl. No.: 15/042,712

(22) Filed: Feb. 12, 2016

Related U.S. Application Data
(63) Continuation of application No. 13/549,057, filed on Jul. 13, 2012, now abandoned.
(60) Provisional application No. 61/507,255, filed on Jul. 13, 2011.

(51) Int. Cl. A61B 5/00; A61B 5/11

(52) U.S. Cl. CPC A61B 5/486; A61B 5/1116; A61B 5/746; A61B 5/7475; A61B 5/6832; A61B 5/6838; A61B 5/6831; A61B 5/742; A61B 5/7405; A61B 5/7455; A61B 5/0022; A61B 5/002; A61B 2562/0219

(57) ABSTRACT

A wireless, programmable biofeedback system, including one or more biofeedback sensor devices, a biofeedback controller device, and a wireless communication link that operationally connects the sensor biofeedback device(s) to the controller biofeedback device. The system may be programmed with desired posture setting, monitor a user's posture with the biofeedback sensor devices for deviations from the desired posture setting, and alert the user to help correct and train to achieve the desired posture setting. Biofeedback controller device may also be, or in addition to, a handheld computer device, such as a smartphone. Further, system may be used to track and graphically represent historical, instant, and predictive future posture records.

### CROSS-REFERENCE TO RELATED APPLICATIONS

This patent application is a continuation application of, and claims priority to, co-pending U.S. patent application Ser. No. 13/549,057, filed Jul. 13, 2012, which claims priority to then co-pending U.S. Provisional Patent Application Ser. No. 61/507,255, filed on Jul. 13, 2011. The disclosures of these foregoing applications are incorporated herein by reference in their entirety.

### TECHNICAL FIELD

The novel technology relates generally to the field of electronic devices, and, more specifically, to a biofeedback system, which may include a related software system, for training people to achieve better posture and other body position goals.

### BACKGROUND

Many activities performed on a frequent basis, such as riding in a vehicle or sitting in a chair, present repeated and unnecessary strains to everyday lives and health. Habituation of bad habits, such as bad posture, further affects health and wellness in both long- and short-term timeframes.

Miniaturization of technologies presents a potential boon to resolving these hindrances. However, most technologies aimed at resolving such everyday hindrances require excessively sized solutions, implantation, medical professional visits, and/or complicated configuration routines. Ultimately, what is needed is a compact, simple solution for kinesthetic awareness and training in one's life.

The present novel technology addresses these needs.

### SUMMARY

The present novel technology relates to personal kinesthetic awareness systems.

The details of one or more embodiments of the subject matter described in this specification are set forth in the accompanying drawings and the description below. Other features, aspects, and advantages of the subject matter will become apparent from the description, the drawings, and the claims.

### BRIEF DESCRIPTION OF THE DRAWINGS

FIG. 1 depicts a schematic diagram of a System of a first embodiment of the present novel technology.

FIG. 2A depicts a schematic representation of a subset of System in the embodiment of FIG. 1.

FIG. 2B depicts a second schematic representation of a subset of System in the embodiment of FIG. 1.

FIG. 3A depicts a first schematic representation of the System of FIG. 1.

FIG. 3B depicts a second schematic representation of the System of FIG. 1.

FIG. 4A depicts screenshots for portions of the System of FIG. 1, more specifically a Home page.

FIG. 4B depicts screenshots for portions of the System of FIG. 1, more specifically an Overview and Instructions page.

FIG. 5A depicts screenshots for portions of the System of FIG. 1, more specifically a Device Status and Settings page.

FIG. 5B depicts screenshots for portions of the System of FIG. 1, more specifically a Sensor Manager page.

FIG. 6 depicts screenshots for portions of the System of FIG. 1, more specifically a Posture and Position Settings page.

FIG. 7A depicts sample screenshots for portions of the System of FIG. 1, more specifically a Biofeedback Alerts page.

FIG. 7B depicts sample screenshots for portions of the System of FIG. 1, more specifically a How to Attach Sensors page.

FIG. 8 depicts sample screenshots for portions of the System of FIG. 1, more specifically a Progress Chart page.

### DETAILED DESCRIPTION

For the purposes of promoting an understanding of the principles of the novel technology and presenting its currently understood best mode of operation, reference will now be made to the embodiments illustrated in the drawings and specific language will be used to describe the same. It will nevertheless be understood that no limitation of the scope of the novel technology is thereby intended, with such alterations and further modifications in the illustrated devices and such further applications of the principles of the novel technology as illustrated therein being contemplated as would normally occur to one skilled in the art to which the novel technology relates. Some embodiments may omit some of the components of system 100, and some embodiments may include other components as well. The illustrated embodiments in the drawings are intended to be exemplary only.

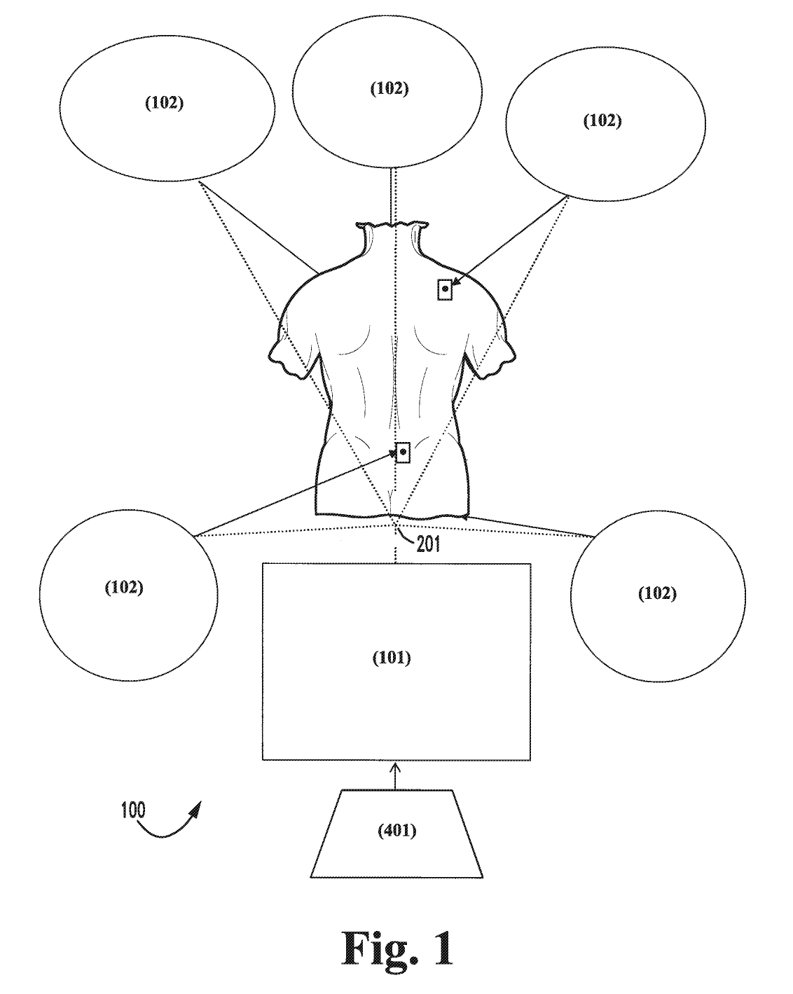

FIG. 1 depicts the present novel technology as a system 100 and its component devices and representative parts of the body monitored for positional changes. System 100 includes one and/or more sensor devices 102 (also referred to as biofeedback sensors, biofeedback sensor devices, and/or sensor biofeedback devices) programmed to detect changes in posture and/or body position through an internal gyroscope and/or based on wireless measurement of the distance between another sensor 102. Each sensor may typically include a built-in vibration, visual, and/or sound device activated by changes in body position (generally also referred to as indicating element and/or indicating device). Such changes are also wirelessly communicated to a remote controller 101, which in some implementations also has built-in sound and/or vibration devices. Controller 101 (also referred to as biofeedback controller, biofeedback control device, and/or controller biofeedback device) may be a specially-made transceiver device, and/or a conventional, programmable device on which system software 401 may be installed (e.g., smartphone, handheld computer, PDA, PC, etc.). Controller 101 maintains a communication link 201 with each sensor, typically via BLUETOOTH (BLUETOOTH is a registered trademark of SIG, Inc., a Delaware corporation, located at 5209 Lake Washington Boulevard, Suite 350, Kirkland, Wash. 98033), infrared, radio frequency, and/or other technologies. Sensors 102 are attached to and/or worn on a person's body (or on clothing and/or accessories) through adhesion, straps, hair and/or other clips, bindings and/or other mechanisms 114. For certain embodiments of system 100, system software 401 may be installed on controller 101, which may display on an internal screen and/or external monitor real-time body position information based on communication from one and/or more sensors. In some implementations, as will be described elsewhere in this application, controller 101 may be a specially made device and/or a conventional computing device (e.g., a smartphone, PDA, laptop, desktop, etc.), which may in turn be programmed with system software 401.

In operation, system 100 may be designed to provide a user with almost immediate feedback about changes in body position and/or posture. Such biofeedback may be used to teach and/or train a user to avoid undesirable, and/or achieve desired, body positions and/or postures. To do so, each sensor 102, typically having the form of a fob-like object, may be actuated automatically to notify the user by an alarm (vibration and/or sound) once either and/or both (i) it deviates from and/or achieves, preset, horizontal and/or vertical planes; and/or (ii) it comes within a preset range of, and/or moves more than a preset distance from, another sensor. Once an alarm is actuated in a sensor, a similar vibration and/or sound alarm may also be wirelessly activated in controller 101, and/or the sound in controller 101 may be programmed to be identical to that of the specific sensor that triggers the initial alarm. By being attached to and/or worn, directly and/or indirectly (e.g., skin, shirt, headband, hairclip, eyeglass frame, etc.), on specific parts of the body, once an alarm is activated in a sensor, the user typically may be able to feel the vibration on and/or about the body part. Sensors 102 may have individualized digital signatures recognized by controller 101 through wireless communication system 201 so as to distinguish it from other sensors that may be part of system 100.

The controller 101 remotely controls the settings for each sensor 102 through wireless communication system 201. Settings may include horizontal and/or vertical planes; sound and/or vibration alarms; distances and/or ranges between sensors; and/or associations of each sensor with a body part. An alarm (audible and/or vibratory) may be set off in controller 101 and/or sensor if either of both (x) a body part to which a sensor is affixed deviates from, and/or achieves, preset horizontal and/or vertical planes; and/or (y) two and/or more paired sensors move beyond, and/or come within, preset ranges and/or distances of each other.

The sensors 102 typically may be small (e.g., the size of a quarter and/or half dollar coin), battery powered programmable transceivers. They may be attached to and/or worn on a user's body and/or clothes through various means, including adhesive backing, straps, hair and/or other clips, and/or bands. Each sensor 102 may be programmed with a distinct alarm sound and/or volume. Devices in system 100 typically may communicate with each other using BLUETOOTH, WI-FI, direct-to-device (D2D) communication protocols (e.g., WI-FI DIRECT (WI-FI DIRECT is a registered trademark of Wi-Fi Alliance, a California corporation, located at 10900-B Stonelake Boulevard, Suite 126, Austin, Tex. 78759); Long Term Evolution (LTE) D2D (LTE is a registered trademark of Institut Europeen des Normes; a French nonprofit telecommunication association, located at 650 route des Lucioles, F-06921, Sophia Antipolis, France), LTE Advanced (LTE-A) D2D, etc.), radio wave, and/or other technologies 201.

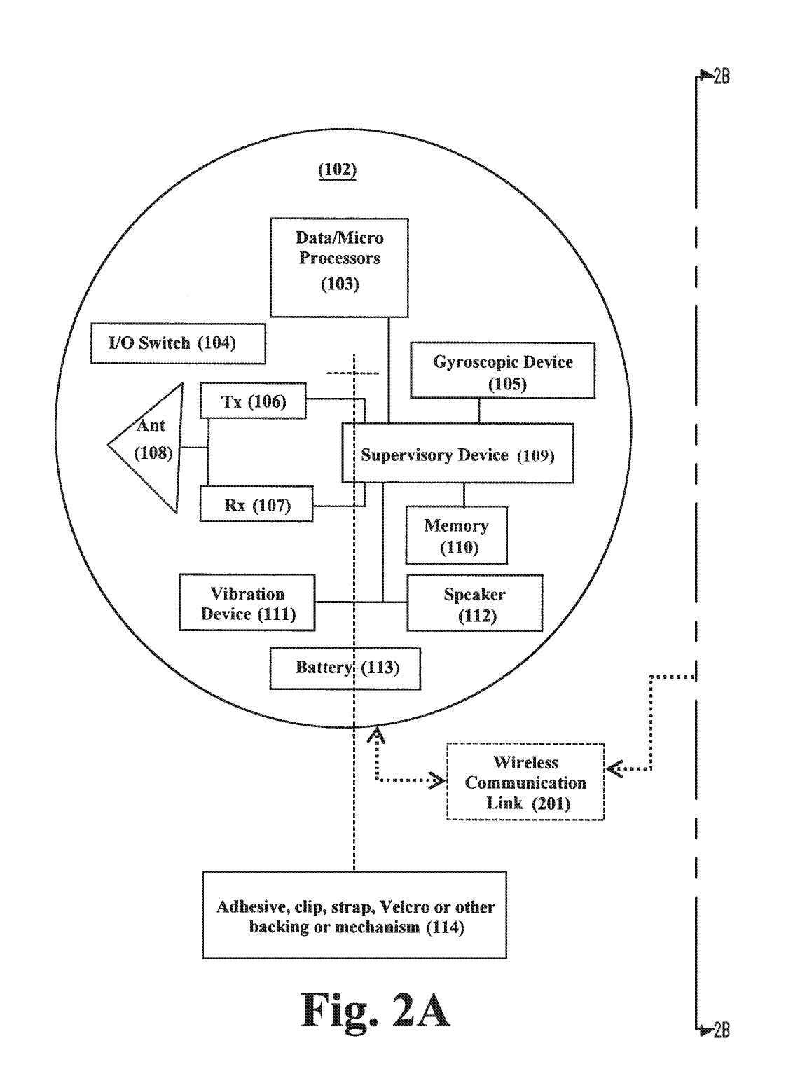

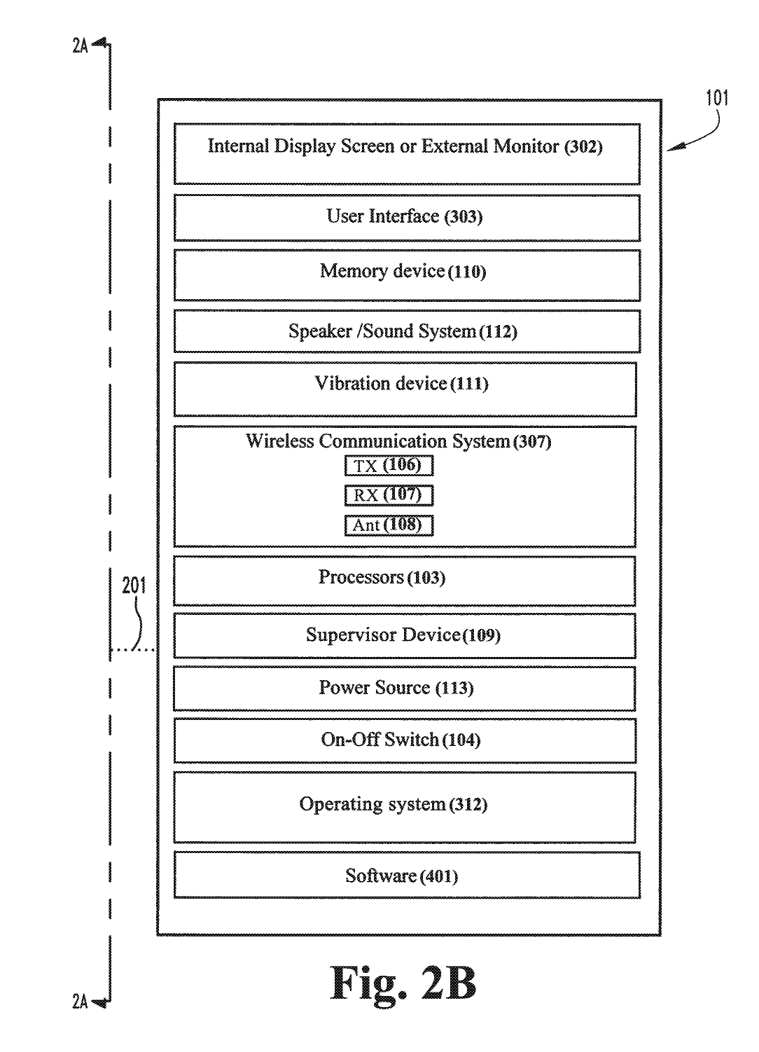

FIG. 2A illustrates certain internal components of the devices included in system 100, which are intended to be exemplary only. As shown in FIG. 2A, each sensor 102 includes a housing containing circuitry and/or other components that may include the following:
(i) A data processor and/or microprocessor 103.
(ii) An on-off switch 104.
(iii) An orientation device 105 (also referred to as detecting element) (e.g., gyroscope, accelerometer, etc.) for detecting and/or communicating pitch, roll, and/or yaw of sensor 102;
(iv) Circuitry for external data communication with controller device 101 and/or in certain embodiments other sensors 102, including a transmitter 106, receiver 107, and/or an antenna 108 that transforms electromagnetic energy to electrical signals provided to receiver 107, and/or transforms electrical signals from transmitter 106 to electromagnetic energy for transmission to remote radio receivers in controller 101 and/or other sensors 102. Receiver 107 responds to the electrical signals from antenna 108 to produce detected data for supervisor device 109. Receiver 107 may include circuits such as filters and/or demodulators. Transmitter 106 responds to formatted data from supervisor device 109 to provide the electrical signals to drive antenna 108. Transmitter 106 may include circuits such as modulators and/or filters. Antenna 108, receiver 107 and/or transmitter 106 together form a radio communication circuit for two-way radio and/or other wireless communication with remote radio devices such as controller 101 and/or other sensors 102.
(v) One or more supervisor devices 109 to control the operation of each sensor 102, which may be implemented as a processor, microprocessor, digital signal processor (DSP), and/or any other logic circuit and/or combination of circuits providing control functions; and/or may operate in response to data and/or to program instructions stored in memory 110; and/or may also control radio and/or other wireless communication circuit components (e.g., 106, 107, 108) by directing the tuning, activation, and/or deactivation of the circuit. In some implementations, supervisor device 109 may be a simple electronic and/or electromechanical circuit interconnecting other system 100 components.
(vi) A memory unit and/or device 110 capable of storing data.
(vii) A vibration device 111 that causes sensor 102 to vibrate.
(viii) A speaker and/or other sound system 112 capable of emitting a variety of sounds (e.g., siren, beep, whistle, gong, etc.).
(ix) A power source 113 (e.g., battery, power supply, capacitor, etc.) and/or conductors 113 to operate sensor 102.
(x) Attachment mechanisms and/or devices 114 by which sensor 102 may be affixed to and/or worn on and/or about a person's body. Such systems and/or devices may include adhesives, hook-and-loop fasteners, like backings, and/or clips.

Also as shown in FIG. 2B, controller 101 may be a device wirelessly linked to and/or and or controlling of the settings/communications of sensor 102. Controller 101 may be a specially-made transceiver device and/or a conventional device (e.g., smartphone, PDA and/or computer) on which software programs 401 may be installed, in either case that may include a housing, circuitry, and/or other components that may include all and/or some of the following:
(i) An internal display screen and/or external monitor 302 enabling a user to view menu options, obtain information about and/or program sensors 102.
(ii) User Interface 303 comprised of a keyboard, keypad, touchscreen, etc. to let user enter data and/or perform programming functions. In some implementations, screen 302 may be user interface 303 (e.g., as a touchscreen).
(iii) A memory unit and/or device 110 capable of storing data.
(iv) A speaker and/or other sound system 112 capable of emitting a variety of sounds (e.g., siren, beep, whistle, gong, etc.).
(v) A vibration device 111 that causes controller 101 to vibrate.
(vi) A wireless communication system 307 (e.g., transmitter 106, receiver 107, and/or antenna 108) for BLUETOOTH, radio wave and/or other communications with each sensor 102.
(vii) A data processor and/or microprocessor 103.
(viii) A supervisor device 109 that may be implemented as a processor, microprocessor, digital signal processor (DSP), and/or any other logic circuit and/or combination of circuits providing control functions. It may operate in response to data and/or to program instructions stored in memory 110, and/or may control radio and/or other wireless communication circuit 307 by directing the tuning, activation, and/or deactivation of the circuit. In some implementations, supervisor device 109 may be a simple electronic and/or electromechanical circuit interconnecting other system 100 components.
(ix) A power source (e.g., battery, line power, capacitors, etc.) and/or conductors 113.
(x) An on-off switch 104.
(xi) Programs and/or operating systems 312 to enable system software and/or application 401 to be installed and/or run on controller 101, which software and/or application may be configured as computer readable program code and/or stored in device's memory 110.

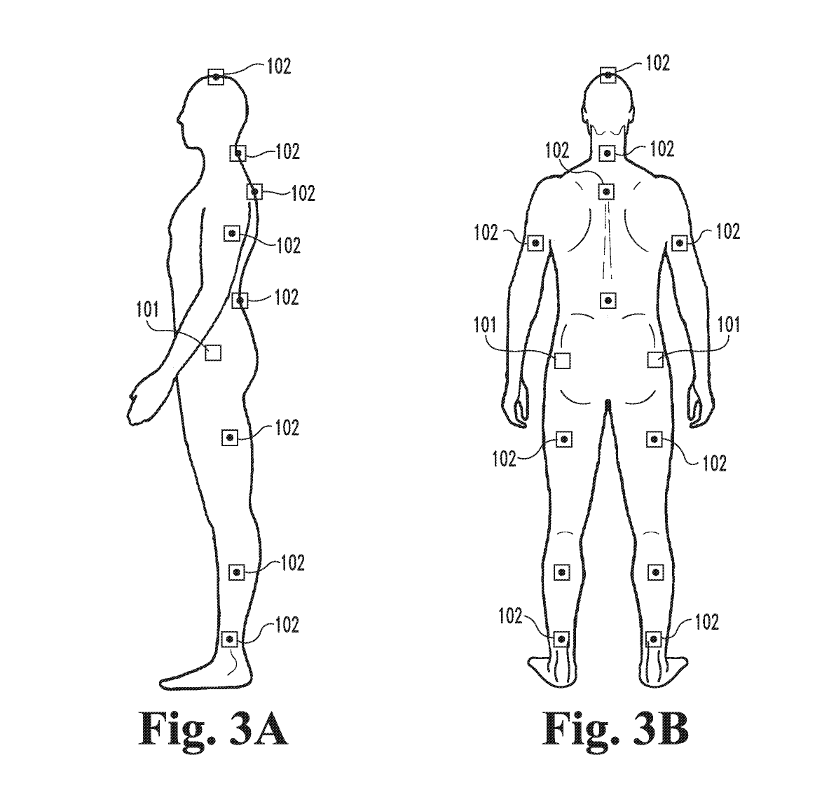

FIGS. 3A-3B illustrates a first embodiment of the present, novel system 100 for training users to attain and/or maintain targeted posture and/or other positions for different parts of the body. The training may be provided through signals (i.e., vibration, sound and/or visual alarms and/or alerts) transmitted to a user and/or activated by measured changes in posture and/or position. Those signals are provided by one and/or more sensors 102 and/or in some embodiments a controller 101. Controller 101 typically may be used to program sensors 102 with posture and/or position parameters (e.g., desired vertical and/or horizontal planes, ranges/distances between sensors 102, etc.). In some embodiments, controller 101 may also illustrate posture and/or body position information through a display, typically based on data from sensors 102 and/or historical records. Each sensor 102 may be directly and/or indirectly (i.e., through clothes and/or accessories) attached to and/or worn on a specific part of a user's body. Those parts of the body typically may be selected based on certain activities the user engages in where biofeedback information and/or training may enhance appearance, performance, health and/or safety. Selected activities may be as simple as standing and/or sitting with head held high and back erect; and/or as complicated as skiing with legs shoulder-width apart, knees bent at a fifty degree angle, and hips centered over the ball of the feet; as critical as keeping a head up and staying awake while driving; and/or the like.

Sensors 102 typically may be placed on the body and/or worn. For example, sensors 102 may be located on an individual's back, skull, neck, boot, foot, leg, chest, hip, and/or the like. Depending on the desired monitoring and/or feedback, one or more sensors 102 may form a mesh to inform controller 101 of various parameters of posture, activity, and/or the like.

The sensor detects and/or activates an alarm when a user attains and/or deviates from a targeted posture and/or position by measuring changes in the user's horizontal and/or vertical fields, and/or by wirelessly measuring distances between two and/or more of such devices. For example, if one sensor is attached to a user's right shoulder, and/or a second is attached to a user's left shoulder, controller 101 sets a target position when both shoulders are arched backward. The coordinates for that target position typically may be represented by either or both (x) the distance between the devices and/or (y) the horizontal/vertical planes of each device. If the user slouches forward, the targeted distance may be exceeded and/or planes change; either of which activates an alarm.

Biofeedback information and/or training may be provided via the alarms from sensors 102 and/or in certain embodiments from controller 101. Those alarms typically may be activated when either (x) the user deviates from a preset position and/or posture (which may be adjusted with tolerances so that a deviation typically may be more than a preset degree (e.g., ten, fifteen, thirty, etc. degrees) and/or for more than a preset period of time (e.g., two, five, twenty, etc. seconds, minutes, hours, etc.) before the alarm is activated); and/or (y) the user achieves a preset, targeted position and/or posture. The devices are programmed so that the alarm typically may continue until a deviation is corrected. By way of example, if a user attaches a sensor 102 to his head and/or neck while driving, vibratory and/or audible alarms typically may be activated in that sensor, and/or in certain embodiments controller, if, having fallen asleep, the user's head tilts so as to deviate by more than fifteen degrees in any direction for more than five seconds from sensor's preset vertical plane. As another example, if a skier has sensors 102 on the front and/or back of a ski boot, and on each hip and/or knee, vibratory and/or audible alarms typically may be activated in each sensor and/or controller 101 once the user attains a preset stance with the right and left feet a distance apart (e.g., one-half, one, two, etc. feet) apart, the knees bent at a fifty degree angle, and/or the hips centered over the heels.

Each sensor 102 typically may have a unique digital signal that allows controller 101, through wireless communication link 201, to program settings for that sensor (e.g., sounds, body position targets, permitted deviations, etc.), and/or to distinguish each sensor from others. Controller 101 has unique digital signals that allow it to communicate with each sensor 102. Controller 101 maintains its communication links to each sensor 102 via a BLUETOOTH, infrared, radio and/or like communication system 201.

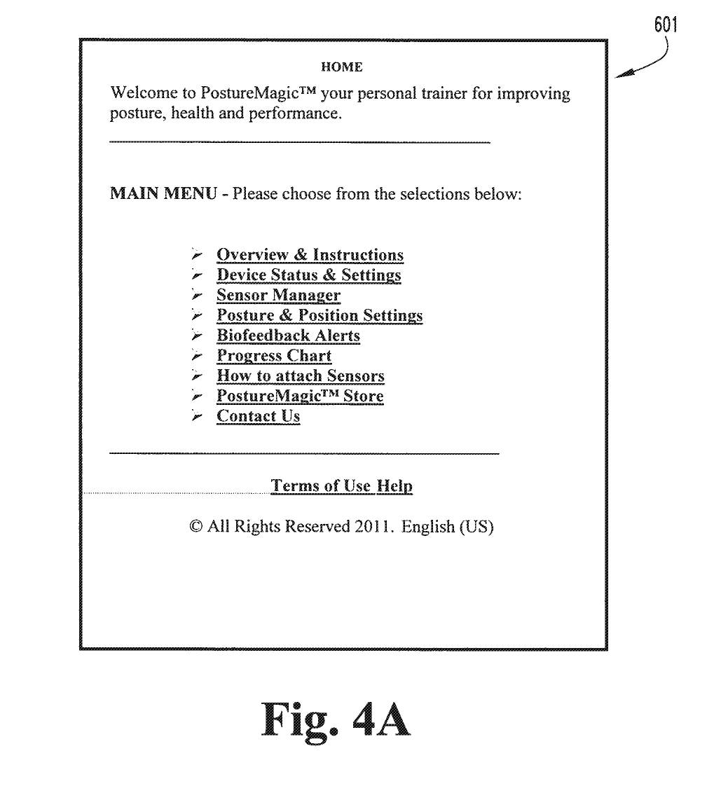

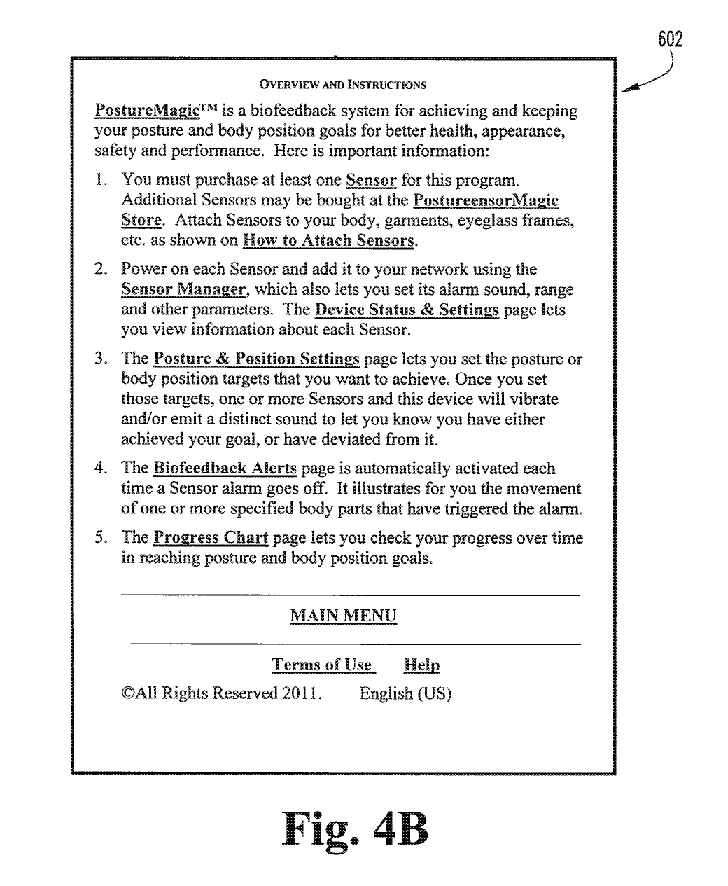

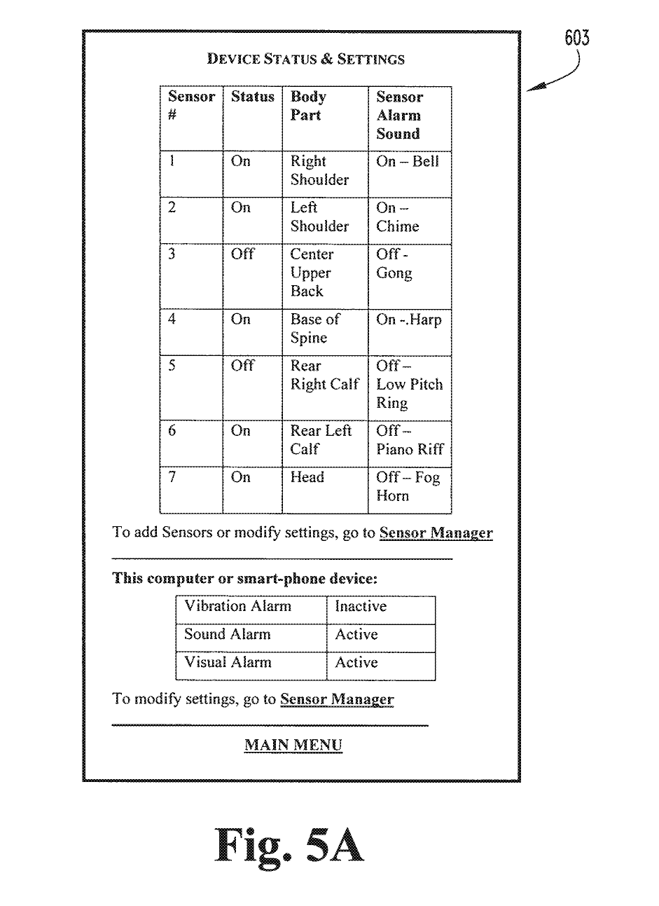

FIGS. 4A-4B illustrates one embodiment of a menu system of, and other screenshots for, system 100 that typically may utilize a device on which system software 401 has been installed (e.g., smartphone, PDA, PC, laptop, etc.). FIG. 4 also illustrates certain functionalities of system 100, and the “look and feel” of system software and/or application 401. It is to be noted that the illustrated menu system and/or screenshots are exemplary only. Other menu systems and/or screenshots/pages may be readily developed and/or provide additional functionalities and/or capabilities.

Home page/main menu 601 typically may be a sample screen/page of system software program/application 401. The menu gives users various options for configuring and/or using system 100 and/or its devices. The selections of subpages accessed through main menu 601 typically are illustrated in 602-608. By clicking on a link to a subpage in main menu 601, that subpage appears on the screen. From any subpage (e.g., 602-608), by clicking on a “Main Menu” link, the user typically may be returned to home page/main menu 601.

System Overview and Instructions screen/page 602 provides the user with brief descriptions of different system 100 components and/or software features. This page typically instructs the user in the operation of system 100 and/or describes its component devices and/or functions. By clicking on any definitional and/or descriptive item highlighted on this page, another subpage opens on the screen with information and/or actions the user may perform.

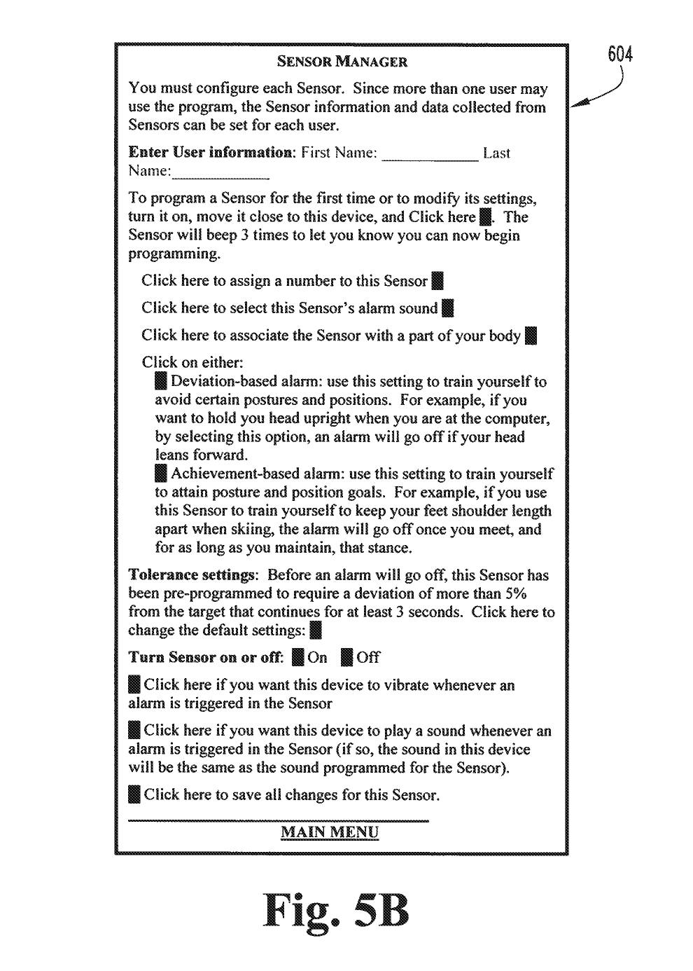

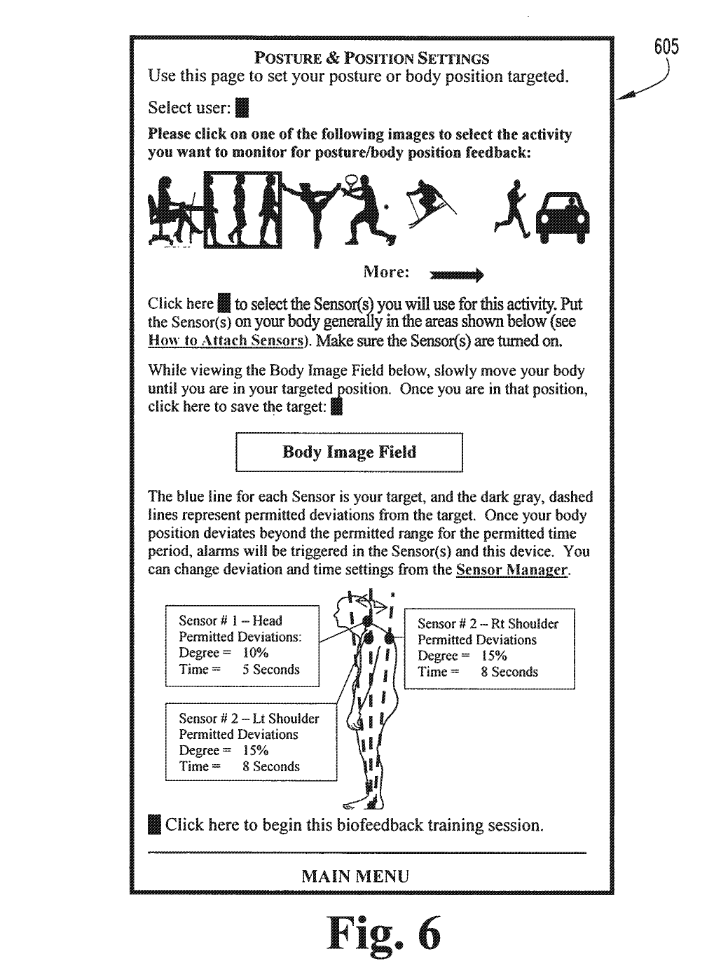

Depicted on FIG. 5A, device status & settings screen/page 603 typically provides the user with a detailed inventory of sensors 102; the body parts/positions with which the sensors are associated; and/or the sound/vibration settings for the sensors and/or controller 101. The page also allows the user to see the status (“on”/“off”) of each sensor, and/or directs the user to modify sensor 102 and/or controller 101 settings and/or add information by clicking on a “Device Manager” link.

For example, sensor “1” may have a status of “On,” be located on the user's right shoulder, and have an enabled sound alarm like a bell. Additionally, sensor “3” may have a status of “Off,” be located on the user's center upper back, and have a sound alarm like a gong. Device status & settings screen/page 603 may also include settings such as vibration, auditory, and/or visual alarm settings and/or state. There may also be links to modify and/or add sensors 102, which typically may redirect the user to device manager screen/page 604.

Depicted on FIG. 5B, device manager screen/page 604 provides various programming functions to manage system devices. Users typically may set parameters for sensors 102 and/or controller 101. Those parameters typically include (i) activating audible alarms in sensors and/or controller, (ii) selecting a specific audible sound for each sensor, (iii) activating a vibration alarm in controller, (iv) setting permitted deviations from targeted positions, (v) designating the period of time a deviation in position may be permitted to continue before an alarm typically may be activated in a sensor, (vi) assigning a number to each sensor, (vii) associating each sensor with a specific part and/or area of the user's body, and/or the like.

For example, device manager screen/page 604 typically may include settings for user information, which sensors 102 are enabled, how sensor 102 may turn on, turn off, provide alarms, and/or customize alarms (e.g., bell, chime, gong, siren, etc.), where the sensor 102 is located, and/or the like. Alarm conditions for sensor 102 alarms may also be configured. For example, deviation-based alarms and/or achievement-based alarms may be configured. Deviation-based alarms typically may be used where a user may wish to avoid certain postures and/or positions, such as holding a head upright. Deviations, for example, may be configured in distance, percentage of deviation (e.g., 1%, 2%, 10%, 50%, etc.) from an expected position, and/or the like. Similarly, achievement-based alarms typically may be used to train a user to attain a posture and/or position. For example, this may trigger an alarm when you sit upright, allowing the user feedback that he or she is properly oriented. Tolerance with these alarms may also be configured, such that sensor 102 typically may not trigger unless a deviation threshold is exceeded.

Depicted on FIG. 6, posture & position screen/page 605 allows users to program targeted positions and/or postures for sensors 102. From this screen, a user may select a prescribed activity (e.g., standing, sitting, driving, skiing, etc.), which may then open a body avatar on which the user may virtually place one and/or more sensors. With a sensor attached to the user's body, he and/or she may then change positions in order to create a targeted posture and/or pose that may be saved. This page/screen also illustrates permitted deviations from targeted positions. Once a targeted position has been saved, the user may proceed to a biofeedback session, and/or typically may be “trained” by receiving vibration and/or sound and/or visual alarms/alerts whenever he and/or she deviates from, and/or achieves, that target.

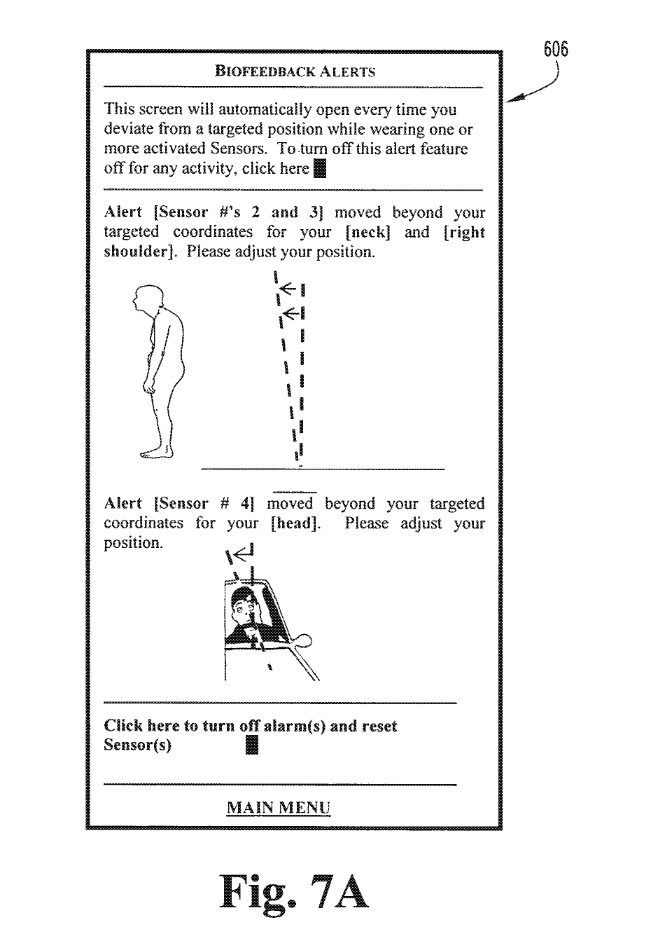

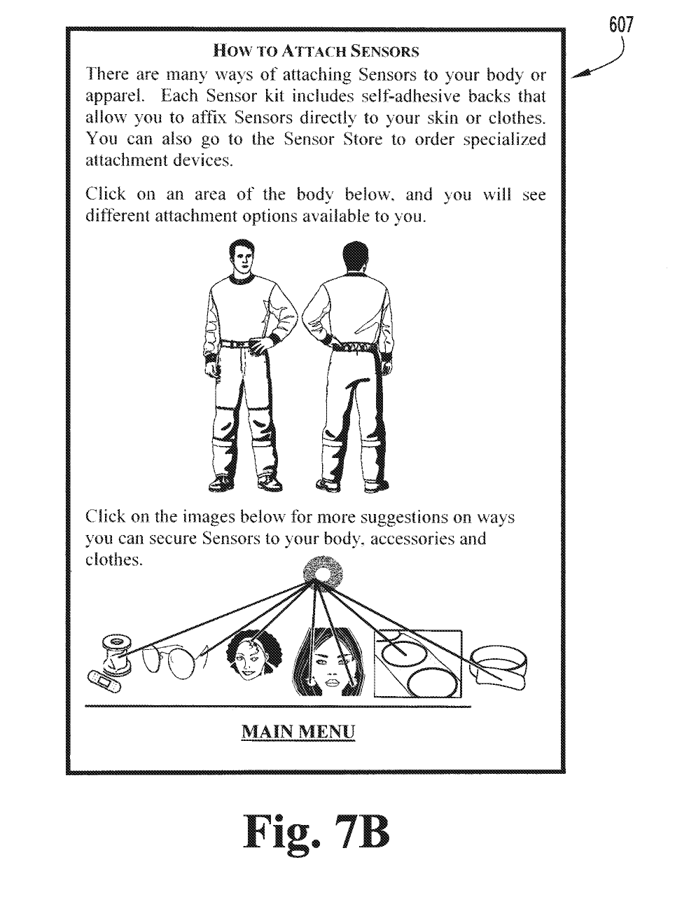

Depicted on FIG. 7A, alerts screen/page 606 provides additional biofeedback information to users. Unless deactivated by a user for an activity, the screen may automatically open each time an alarm typically may be activated by one and/or more sensors 102. The screen typically provides real-time notifications and/or visual displays of deviations from, and/or attainment of, targeted postures and/or positions. Alerts screen/page 606 typically may also allow a system 100 user to turn off alerts, customize alert deviation threshold, reset sensors 102, and/or the like. In some further implementations, selections may be provided for saved activities and/or postures. For example, a user may select from a drop-down list of sports, postures, and/or combinations thereof for one or more body parts to customize and/or set system 100 parameters. In yet further implementations, these saved selections may also enable and/or disable one or more sensors 102 temporarily and/or permanently so as to cease alarms that may otherwise be generated.

Depicted on FIG. 7B, How to Attach Sensors screen/page 607 provides instructions for attaching sensors 102 to parts of the user's body. A user typically may be instructed how sensors 102 should be attached to body parts, clothing, accessories, and/or the like. In some implementations, selecting one or more body parts on How to Attach Sensors screen/page 607 (i.e., by clicking on an image, selecting from a list, and/or the like) may generate one or more instruction routines for attachment to that selection.

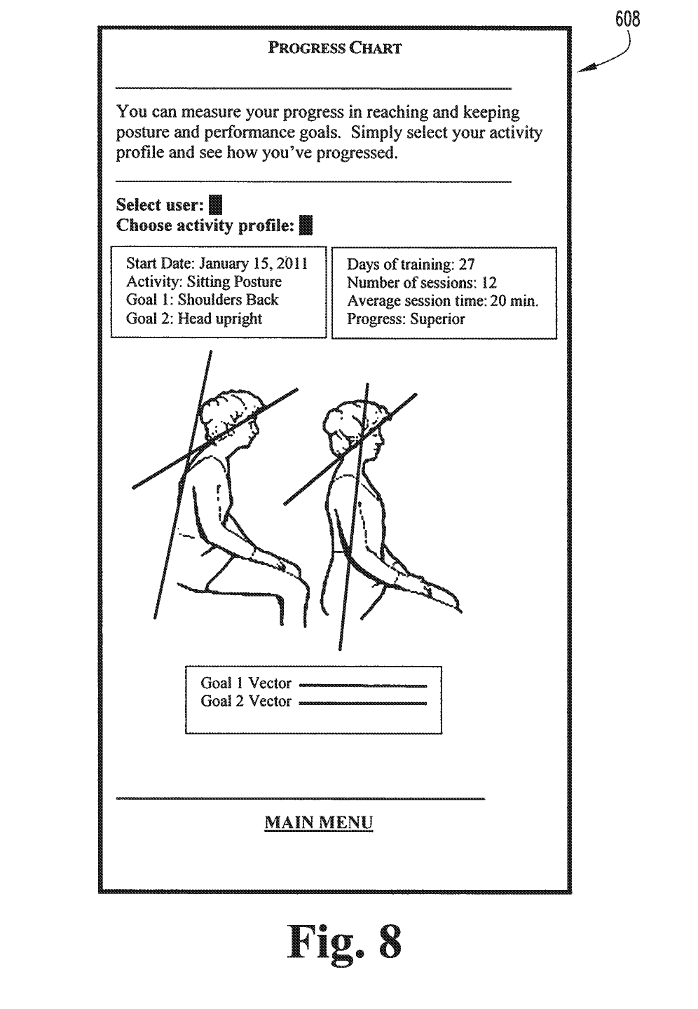

As depicted on FIG. 8, Progress Chart screen/page 608 typically may provide historical and/or real-time data to a user about progress in reaching and/or maintaining targeted posture and/or position goals. From this screen/page 608, a user may select the activity he and/or she wishes to monitor, and/or may view a graphic representation of progress in relation to the established target for that activity. For example, a user may be able to view his or her posture history graphically and/or statistically as a series of images, graphic, video, and/or the like.

Such display may, in some implementations, allow predictive displays as well. For example, based on a user's progress historically, the system may analyze and calculate to predict (numerically, graphically, and/or the like) how a user will look at a given timeframe and/or when a user may reach a certain posture/position. In certain embodiments, a user may record a real-time representation of posture/position changes during an activity and/or play the recording back to gauge progress.

While the novel technology has been illustrated and described in detail in the drawings and foregoing description, the same is to be considered as illustrative and not restrictive in character. It is understood that the embodiments have been shown and described in the foregoing specifications in satisfaction of the best mode and enablement requirements. It is further understood that one of ordinary skill in the art could readily make a nigh-infinite number of insubstantial changes and modifications to the above-described embodiments and that it would be impractical to attempt to describe all such embodiment variations in the present specification. Accordingly, it is understood that all changes and modifications that come within the spirit of the novel technology are desired to be protected.

While a particular embodiment of the present invention has been shown and described, modifications may be made. It is therefore intended in the appended claims to cover such changes and modifications which follow in the true spirit and scope of the invention.

What is claimed is:

1. A personal kinesthetic awareness system for notifying and training a user, comprising:
at least one sensor biofeedback device, the at least one sensor biofeedback device comprising:
a first housing at least partially defining the bounds of the at least one sensor biofeedback device;
a sensor biofeedback device supervisor located at least partially within the first housing;
a first transceiver operationally connected to the sensor biofeedback device supervisor;
a first processor operationally connected to the sensor biofeedback device supervisor;
at least one detecting element operationally connected to the sensor biofeedback device supervisor;
a first memory operationally connected to the sensor biofeedback device supervisor, wherein the first memory stores a first predetermined signal;
a first electrical power source operationally connected to the sensor biofeedback device supervisor; and
at least one attachment element operationally connected the first housing and attachable to at least one attachment location;
at least one controller biofeedback device; and
a wireless communication link that operationally connects the at least one sensor biofeedback device to the at least one controller biofeedback device to form a wireless communications system;
wherein the at least one sensor biofeedback device detects changes in posture based on the at least one attachment location and operationally communicates the changes wirelessly to the at least one controller biofeedback device;
wherein the at least one controller biofeedback device trains a user to achieve at least one body position; and
wherein the at least one sensor biofeedback device and the at least one controller biofeedback device are not implanted in a user's body.

2. The personal kinesthetic awareness system of claim 1:
wherein the at least one body position is set, monitored, and modified;
wherein the at least one controller biofeedback device stores the wirelessly communicated changes in posture for a historical posture record;
wherein the historical posture record represents an extended time period of at least about a week;
wherein progress toward the at least one body position is monitored by viewing the historical posture record as a pictorial representation of the user's posture; and
wherein the at least one sensor biofeedback device and the at least one controller biofeedback device are not implanted in a user's body.

3. The personal kinesthetic awareness system of claim 1, wherein the at least one controller biofeedback device comprises:
a second housing at least partially defining the bounds of the at least one controller biofeedback device;
a controller biofeedback device supervisor located at least partially within the second housing;
a second transceiver operationally connected to the controller biofeedback device supervisor;
a second processor operationally connected to the controller biofeedback device supervisor;
at least one user interface element operationally connected to the controller biofeedback device supervisor;
a second memory operationally connected to the controller biofeedback device supervisor, wherein the second memory stores a second predetermined signal; and
a second electrical power source operationally connected to the controller biofeedback device supervisor.

4. The personal kinesthetic awareness system of claim 1, wherein the at least one controller biofeedback device is a smartphone.

5. The personal kinesthetic awareness system of claim 1, wherein the at least one detecting element is selected from the group consisting of gyroscopic devices, distance measuring devices, and combinations thereof.

6. The personal kinesthetic awareness system of claim 1, wherein the at least one sensor biofeedback device further comprises:
at least one first alarm element operationally connected to the sensor biofeedback device supervisor.

7. The personal kinesthetic awareness system of claim 3, wherein the at least one controller biofeedback device further comprises:
at least one second alarm element operationally connected to the controller biofeedback device controller.

8. The personal kinesthetic awareness system of claim 1, wherein the at least one attachment element is selected from the group consisting of adhesives, hook-and-loop fasteners, clips, straps, and bindings.

9. The personal kinesthetic awareness system of claim 1, wherein the at least one body position is set based on an instant posture of the user's body detected by the sensor biofeedback device.

10. A kit for detecting and correcting posture, the kit comprising:
at least one sensor device, the at least one sensor device comprising:
a first housing at least partially defining the bounds of the at least one sensor device;
a sensor device supervisor located at least partially within the first housing;
a first transceiver operationally connected to the sensor device supervisor;
a first processor operationally connected to the sensor device supervisor;
at least one second detecting element operationally connected to the sensor device supervisor;
a first memory operationally connected to the sensor device supervisor, wherein the first memory stores a first predetermined signal;
a first electrical power source operationally connected to the sensor device supervisor; and
at least one attachment element operationally connected the first housing and attachable to at least one attachment location;
at least one controller device, wherein the at least one controller device is wirelessly connectable to the at least one sensor device;
wherein the at least one sensor device detects changes in posture based on the at least one attachment location and operationally communicates the changes wirelessly to the at least one controller device;
wherein the at least one controller device trains a user to achieve at least one body position; and
wherein the at least one sensor device and the at least one controller device are not implanted in a user's body.

11. The kit of claim 10, wherein the at least one controller device comprises:
a second housing at least partially defining the bounds of the at least one controller device;
a controller device supervisor located at least partially within the second housing;
a second transceiver operationally connected to the controller device supervisor;
a second processor operationally connected to the controller device supervisor;
at least one user interface element operationally connected to the controller device supervisor;
at least one indicating element operationally connected to the controller device supervisor,
a second memory operationally connected to the controller device supervisor, wherein the second memory stores a second predetermined signal, and
a second electrical power source operationally connected to the controller device supervisor;
wherein the at least one indicating element trains the user to achieve the at least one body position.

12. The kit of claim 10, wherein the at least one second detecting element is selected from the group consisting of gyroscopic devices, distance measuring devices, and combinations thereof.

13. The kit of claim 10, wherein the at least one sensor device further comprises:
at least one first alarm element operationally connected to the sensor device supervisor.

14. The kit of claim 11, wherein the at least one controller device further comprises:
at least one second alarm element operationally connected to the controller device controller.

15. The kit of claim 10, wherein the at least one attachment element is selected from the group consisting of adhesives, hook-and-loop fasteners, clips, straps, and bindings.

16. The kit of claim 10, wherein the at least one body position is set based on an instant posture of the user's body detected by the sensor device.

17. A nontransitory computer-readable storage medium comprising instructions to cause one or more processors to:
receive target posture parameters from at least one user input device;
monitor at least one sensor biofeedback device for sensed posture data;
wirelessly communicate the sensed posture data between the at least one sensor biofeedback device and a controller biofeedback device;
compare the sensed posture data with the target posture parameters;
provide at least one alarm if the sensed posture data does not match the target posture parameters within a predetermined window;
store the sensed posture data over a period of time;
compare the stored posture data to the target posture parameters over the period of time;
prepare the compared posture data for display to a user as a historical posture record, wherein the historical posture record represents an extended time period of at least about a week, wherein the historical posture record pictorially depicts the user's posture over the extended time period based on the sensed posture data, the stored posture data, and the compared posture data; and
display the compared posture data and the historical posture record to the user on at least one display element as an illustrated representation of the user's posture, wherein the at least one display element is selected from the group consisting of a touchscreen, an external monitor, and an internal monitor;
wherein the at least one sensor biofeedback device and the controller biofeedback device are not implanted in a user's body.

18. The nontransitory computer-readable storage medium of claim 17, which further causes the one or more computers to:
cease the at least one alarm once the sensed posture data matches the target posture parameters.

19. The nontransitory computer-readable storage medium of claim 18, wherein the predetermined window is variable based on preset deviations selected from the group consisting of degree of posture deviation from the target posture parameters, length of time deviating from the target posture parameters, and combinations thereof.

20. The nontransitory computer-readable storage medium of claim 17, wherein the at least one alarm is emitted from a member of a group selected from the at least one sensor biofeedback device, the controller biofeedback device, and combinations thereof.
